(19) United States
(12) Patent Application Publication
(10) Pub. No.: US 2026/0115451 A1
(43) Pub. Date: Apr. 30, 2026

(54) BOWL-SHAPED PERMANENT MAGNET BRUSHLESS DIRECT-CURRENT MOTOR AND CONTROL METHOD THEREFOR AND ARTIFICIAL HEART PUMP

(71) Applicant: SHANDONG UNIVERSITY, Jinan (CN)

(72) Inventors: Wenliang ZHAO, Jinan (CN); Ning WANG, Jinan (CN); Bitan WANG, Jinan (CN); Zhongkun CAO, Jinan (CN); Cong LIU, Jinan (CN); Qiyu GUO, Jinan (CN); Xian'ao WANG, Jinan (CN)

(21) Appl. No.: 19/303,487

(22) Filed: Aug. 19, 2025

(30) Foreign Application Priority Data
Oct. 25, 2024 (CN) 2024115038518

(51) Int. Cl. A61M 60/546; A61M 60/232; A61M 60/419; A61M 60/422; H02K 1/16; H02K 1/274; H02P 6/182; H02P 21/05; H02P 21/13

(52) U.S. Cl. CPC A61M 60/546; A61M 60/419; A61M 60/422; H02K 1/165; H02P 6/182; H02P 21/05; H02P 21/13; A61M 60/232; H02K 1/274; H02P 2207/05

(57) ABSTRACT

A bowl-shaped permanent magnet brushless direct-current motor, including a bowl-shaped stator core and a plurality of permanent magnets; wherein, the bowl-shaped stator core includes a stator yoke portion and a plurality of bent teeth; a height difference exists between a tooth shoe of the bent tooth and a plane on which the stator yoke portion is located, and the tooth shoe of the bent tooth is located at the same height as the permanent magnet in an axial direction, so that an armature magnetic flux path is located at the same height as a permanent magnet excitation magnetic flux path, thereby implementing an unobstructed magnetic flux path.

### CROSS-REFERENCE TO RELATED APPLICATIONS

The present disclosure claims priority to Chinese Patent Application No. 202411503851.8, filed with the China National Intellectual Property Administration on Oct. 25, 2024 and entitled “Bowl-Shaped Permanent Magnet Brushless Direct-current Motor and Control Method Therefor and Artificial Heart Pump”, which is incorporated herein by reference in its entirety and constitutes a part of the present disclosure for all purposes.

### TECHNICAL FIELD

The present disclosure relates to the technical field of artificial heart pumps, and in particular, to a bowl-shaped permanent magnet brushless direct-current motor and a control method therefor and an artificial heart pump.

### BACKGROUND

The statements in this section only provide background related to the present disclosure and do not necessarily constitute the prior art.

Permanent magnet brushless direct-current motors are widely applied to the field of medical instruments across a wide range of power levels because of its simple structure, high efficiency, high power density, long service life and good mechanical properties, but higher requirements are also put forward for the permanent magnet brushless direct-current motors.

An artificial heart pump has a compact mechanical structure. Due to internal space limitations, a novel artificial heart pump requires that the stator and the rotor of the permanent magnet brushless direct-current motor are not in the same plane. In a conventional permanent magnet brushless direct-current motor, the rotor, the permanent magnet, the stator and the winding are all at the same level, so the conventional permanent magnet brushless direct-current motor is difficult to adapt to the novel artificial heart pump, which limits the application and popularization of the artificial heart pump.

In addition, the permanent magnet of an existing permanent magnet brushless direct-current motor is mainly tile-shaped, and the torque performance is not optimal in the face of the actual working condition of a wide air gap of the artificial heart pump. However, a conventional eccentric permanent magnet is mainly applied to a permanent magnet synchronous motor to improve the sine of the air gap of the permanent magnet synchronous motor, and is not applicable to the permanent magnet brushless direct-current motors.

### SUMMARY

A technical problem to be solved by the present disclosure lies in how to design a bowl-shaped permanent magnet brushless direct-current motor to achieve optimal torque performance under load working conditions.

To address the shortcomings of the prior art, the present disclosure provides a bowl-shaped permanent magnet brushless direct-current motor and a control method therefor and an artificial heart pump. The geometric structure of the bowl-shaped permanent magnet brushless direct-current motor is different from that of a conventional motor, and teeth of a bowl-shaped stator core are bent upward, so that an armature magnetic flux path is located at the same height as a permanent magnet excitation magnetic flux path, thereby implementing an unobstructed magnetic flux path. The permanent magnet brushless direct-current motor is suitable for artificial heart pumps and can operate stably and efficiently, and solves the problem that an artificial heart pump and a permanent magnet brushless direct-current motor thereof are difficult to adapt in the prior art.

To achieve the above purpose, the following technical solutions are employed in the present disclosure:

According to a first aspect of the present disclosure, a bowl-shaped permanent magnet brushless direct-current motor is provided.

The bowl-shaped permanent magnet brushless direct-current motor includes a bowl-shaped stator core and a plurality of permanent magnets.

The bowl-shaped stator core includes a stator yoke portion and a plurality of bent teeth. A height difference exists between a tooth shoe of the bent tooth and a plane on which the stator yoke portion is located, and the tooth shoe of the bent tooth is located at the same height as the permanent magnet in an axial direction, so that an armature magnetic flux path is located at the same height as a permanent magnet excitation magnetic flux path.

Further, the permanent magnet is a non-uniform thickness permanent magnet. A radial outer edge of the non-uniform thickness permanent magnet is an arc, and a circle center of the outer edge arc is on a rotation axis of the motor. A radial inner edge of the non-uniform thickness permanent magnet is also an arc, a circle center of the inner edge arc is not on the rotation axis of the motor, and a radius of the inner edge arc is less than a maximum distance between any point of the inner edge and the circle center of the outer edge arc.

Further, the bowl-shaped permanent magnet brushless direct-current motor further includes a rotor core. A radial outer edge of the rotor core coincides with the radial inner edge of the non-uniform thickness permanent magnet to form a plum blossom-shaped structure.

Further, the non-uniform thickness permanent magnets are evenly distributed in a circumferential direction of the rotor core, and adjacent non-uniform thickness permanent magnets are alternately magnetized inwards and outwards in a radial direction.

Further, the bowl-shaped permanent magnet brushless direct-current motor further includes a stator winding. The stator winding is wound around the bent teeth.

According to a second aspect of the present disclosure, a control method for a bowl-shaped permanent magnet brushless direct-current motor is provided.

The control method for the bowl-shaped permanent magnet brushless direct-current motor according to the first aspect includes the following steps:
calculating a control voltage reference value by means of an active disturbance rejection controller and a hysteresis current controller, and controlling the bowl-shaped permanent magnet brushless direct-current motor according to the first aspect by means of a three-phase inverter.

Further, the active disturbance rejection controller obtains an output of the active disturbance rejection controller based on a deviation between a given rotational speed value and an observed rotational speed value.

Further, steps of calculating the observed rotational speed value include:
acquiring a voltage output value and a current output value;
based on the voltage output value and the current, obtaining a back electromotive force value by means of back electromotive force detection; and
based on the back electromotive force value, obtaining the observed rotational speed value by means of speed processing and calculation.

Further, the active disturbance rejection controller estimates disturbance of the bowl-shaped permanent magnet brushless direct-current motor by employing an extended state observer.

According to a third aspect of the present disclosure, an artificial heart pump is provided.

The artificial heart pump employs the bowl-shaped permanent magnet brushless direct-current motor according to the first aspect.

Compared with the prior art, the present disclosure has the following beneficial effects:

The geometrical structure of the bowl-shaped permanent magnet brushless direct-current motor of the present disclosure is different from that of a conventional motor, and teeth of the bowl-shaped stator core are bent upward, so that an armature magnetic flux path is located at the same height as a surface-mounted permanent magnet excitation magnetic flux path, thereby implementing an unobstructed magnetic flux path. The permanent magnet brushless direct-current motor is suitable for artificial heart pumps and can operate stably and efficiently, and solves the problem that an artificial heart pump and a permanent magnet brushless direct-current motor thereof are difficult to adapt in the prior art.

In the bowl-shaped permanent magnet brushless direct-current motor of the present disclosure, the non-uniform thickness permanent magnet is thin in the middle and thick at two ends, so that the impact brought by the wide air gap of the artificial heart pump and the interpolar magnetic flux leakage can be reduced, thereby improving the torque performance.

### BRIEF DESCRIPTION OF THE DRAWINGS

The accompanying drawings of the specification, which constitute a part of the present disclosure, are used for providing a further understanding of the present disclosure. The illustrative embodiments of the present disclosure and descriptions thereof are used for explaining the present disclosure and do not constitute an improper limitation of the present disclosure.

FIG. 1 is a three-dimensional (3D) structure diagram of a bowl-shaped permanent magnet brushless direct-current motor according to Example 1 of the present disclosure;

FIG. 2 is a 3D sectional view of the bowl-shaped permanent magnet brushless direct-current motor according to Example 1 of the present disclosure;

FIG. 3 is a two-dimensional plan view of a rotor of the bowl-shaped permanent magnet brushless direct-current motor according to Example 1 of the present disclosure;

FIG. 4 is a load torque waveform diagram of the bowl-shaped permanent magnet brushless direct-current motor according to Example 1 of the present disclosure;

FIG. 5 is a flowchart of a control method for a bowl-shaped permanent magnet brushless direct-current motor according to Example 2 of the present disclosure;

FIG. 6 is a structure diagram of an active disturbance rejection controller according to Example 2 of the present disclosure; and

FIG. 7 is a schematic diagram of an artificial heart pump according to Example 3 of the present disclosure.

### DETAILED DESCRIPTION

The present disclosure is further described below in conjunction with the accompanying drawings and embodiments.

It should be noted that the following detailed descriptions are exemplary, and are intended to further describe the present disclosure. Unless otherwise specified, all technical and scientific terms used herein have the same meanings as those commonly understood by those of ordinary skill in the art to which the present disclosure belongs.

It should be noted that the terms used herein are only for describing specific implementations, and are not intended to limit exemplary implementations according to the present disclosure. As used herein, unless otherwise explicitly specified in the context, the singular form is also intended to include the plural form. In addition, it should be understood that when the terms “include/including” and/or “comprise/comprising” are used in this specification, they indicate the presence of features, steps, operations, devices, components and/or combinations thereof.

In the present disclosure, the azimuth or positional relationship indicated by terms such as “up”, “down”, “left”, “right”, “front”, “back”, “vertical”, “horizontal”, “side”, and “bottom” is based on the azimuth or positional relationship shown in the accompanying drawings, and is only determined for the purpose of facilitating the description of the structural relationship of each part or element of the present disclosure, and does not specifically refer to any part or element in the present disclosure, and cannot be understood as a limitation on the present disclosure.

In the present disclosure, terms such as “fixedly connected”, “connected to each other”, and “connected” should be understood in a broad sense and indicate that it may be fixedly connected, or may be integrally connected or detachably connected; may be directly connected, or may be indirectly connected via an intermediate medium. For a relevant scientific or technical person in the art, specific meanings of the foregoing terms in the present disclosure may be determined according to specific situations, and should not be understood as limitations to the present disclosure.

Without conflict, embodiments of the present disclosure and features of the embodiments may be combined with each other.

### Example 1

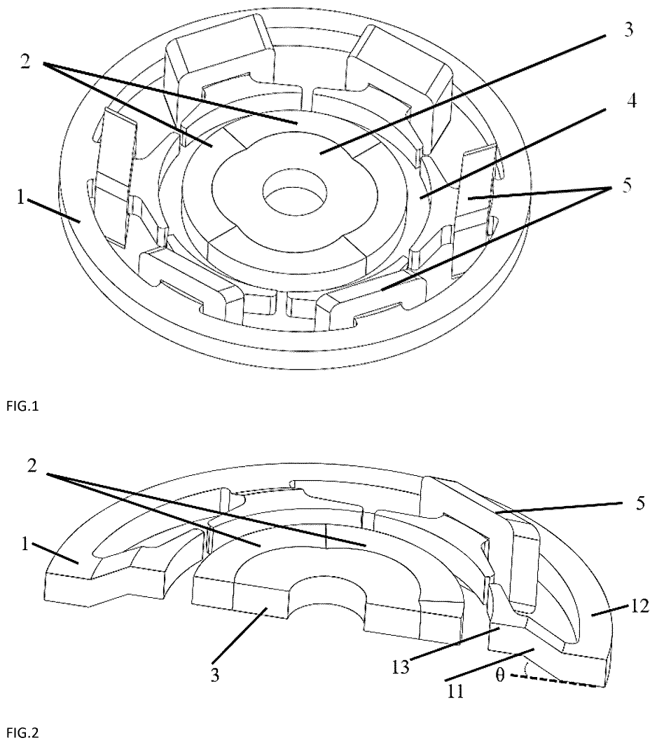

Example 1 of the present disclosure provides a bowl-shaped permanent magnet brushless direct-current motor, which is applicable to an artificial heart pump. As shown in FIG. 1 and FIG. 2, the bowl-shaped permanent magnet brushless direct-current motor includes a stator and a rotor, and an air gap 4 is formed between the stator and the rotor.

The number of slots of the stator is 6, the number of poles of a non-uniform thickness permanent magnet 2 is 4, and the stator includes a bowl-shaped stator core 1 and a stator winding 5.

The bowl-shaped stator core 1 includes a stator yoke portion 12 and 6 bent teeth 11. In the present embodiment, the bent tooth 11 and the stator yoke portion 12 form an angle θ in space. The bending angle θ in the present example is 30°.

To make a magnetic path unobstructed, all the bent teeth 11 have the same bending angle. Therefore, axial upper planes of tooth shoes 13 of the bent teeth 11 are coplanar and axial lower planes are coplanar, and are parallel to a plane on which the stator yoke portion 12 is located.

In other words, the plane on which the stator yoke portion 12 is located is parallel with the horizontal plane; the bent tooth 11 is composed of a ramp-shaped component and the tooth shoe 13; an included angle between the ramp-shaped component and the horizontal plane is θ; and the tooth shoe 13 is parallel with the plane on which the stator yoke portion 12 is located.

The stator winding 5 is wound around the bent teeth 11 inclined in space to supply power to the bowl-shaped permanent magnet brushless direct-current motor, thereby driving the artificial heart pump to supply blood.

A lamination of the bowl-shaped stator core 1 may be formed by secondary stamping and bending of a lamination of an existing planar stator core. Due to the limitations of the motor structure of the artificial heart pump, a thickness of the air gap 4 of the bowl-shaped permanent magnet brushless direct-current motor is greater than half of an axial length of the motor, thereby causing a permanent magnet-magnetic chain to have large magnetic flux leakage in the circumferential direction.

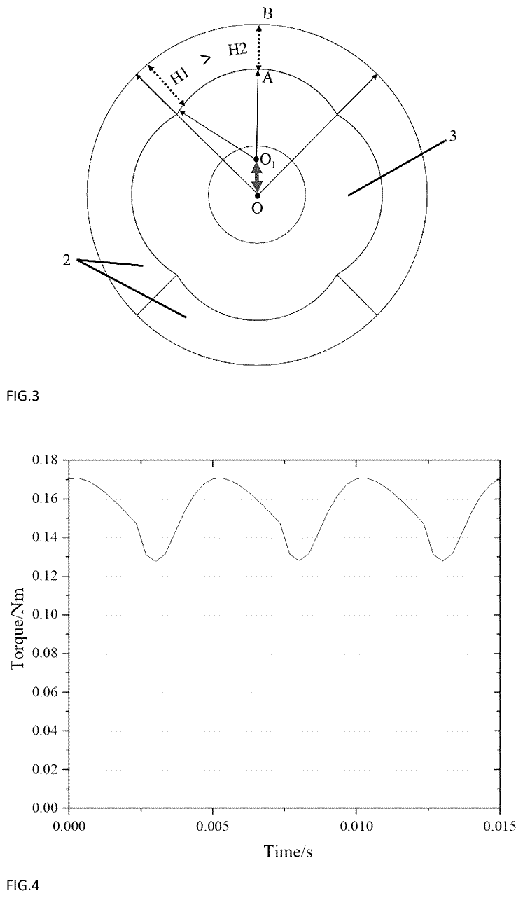

As shown in FIG. 3, the rotor of the bowl-shaped permanent magnet brushless direct-current motor includes a plurality of non-uniform thickness permanent magnets 2 of a consistent shape and size and a rotor core 3. A radial outer edge of each non-uniform thickness permanent magnet 2 is an arc, and a circle center O of the outer edge arc is on a rotation axis of the motor. A radial inner edge of the non-uniform thickness permanent magnet 2 is also an arc, a circle center O₁ of the inner edge arc is not on the rotation axis of the motor, and a radius of the inner edge arc is less than a maximum distance between any point of the inner edge and the point O. An O₁O straight line intersects with the non-uniform thickness permanent magnet 2 at a line segment AB, and a length H2 of the line segment AB equals to the smallest radial thickness of the non-uniform thickness permanent magnet 2, and is less than any radial thickness H1 of the non-uniform thickness permanent magnet 2. The non-uniform thickness permanent magnet 2 is thin in the middle and thick at both ends, so that the impact brought by the wide air gap of the artificial heart pump and the interpolar magnetic flux leakage can be compensated and reduced, thereby improving the torque performance. The motor performance is shown in FIG. 4, and the average torque is 0.1546 Nm.

It should be noted that the arc mentioned in the present example is a shape of a part of a circle.

The non-uniform thickness permanent magnets 2 are evenly distributed on the rotor in a circumferential direction, and adjacent non-uniform thickness permanent magnets 2 are alternately magnetized inwards and outwards in a radial direction.

A radial outer edge of the rotor core 3 coincides with the radial inner edge of the non-uniform thickness permanent magnet 2 to form a plum blossom-shaped structure.

The tooth shoe 13 of the bowl-shaped stator core 1 is at the same height as the non-uniform thickness permanent magnet 2 in an axial direction. A height difference exists between the tooth shoe and the plane on which the stator yoke portion 12 is located in the axial direction, so that an armature magnetic flux path can be at the same height as an excitation magnetic flux path of the non-uniform thickness permanent magnet 2, thereby implementing an unobstructed magnetic flux path, such that the permanent magnet brushless direct-current motor is suitable for artificial heart pumps and can operate stably and efficiently, and solves the problem that an artificial heart pump and a permanent magnet brushless direct-current motor thereof are difficult to adapt in the prior art.

### Example 2

The present embodiment provides a control method for the bowl-shaped permanent magnet brushless direct-current motor according to Example 1. The control method is a position-sensorless control method. Position-sensorless control refers to that the motor is controlled without the help of a “position sensor”. As shown in FIG. 5, the control method includes the following steps:
step 1: acquire a given rotational speed value ω*, and subtract an observed rotational speed value ω from the given rotational speed value ω* to obtain a rotational speed deviation ω*-ω;
step 2: input the rotational speed deviation ω*-ω into an active disturbance rejection controller to obtain a compensated output I* of the active disturbance rejection controller;
step 3: based on the output I* and a current output value I_{abc} of the active disturbance rejection controller and an output of commutation logic calculation, control an output (control voltage reference value) of a hysteresis current controller, and further control the bowl-shaped permanent magnet brushless direct-current motor M according to Example 1 by means of a three-phase inverter, and finally, measure a voltage output value U_{abc} and a current output value I_{abc} of each phase winding of the bowl-shaped permanent magnet brushless direct-current motor by means of a voltmeter/sensor and an ammeter/sensor;
step 4: based on the voltage output value U_{abc} and the current output value I_{abc}, obtain a back electromotive force value e_{abc} by means of back electromotive force detection;
step 5: based on the back electromotive force value e_{abc}, obtain an output of the commutation logic calculation by means of commutation logic calculation; and
step 6: based on the back electromotive force value e_{abc}, obtain the observed rotational speed value ω by means of speed processing and calculation.

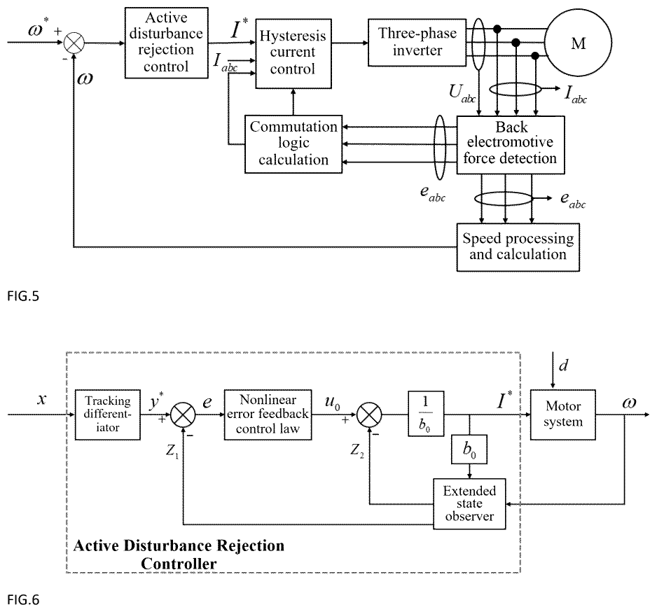

For the active disturbance rejection controller in step 2, as shown in FIG. 6, the active disturbance rejection controller includes a tracking differentiator, a nonlinear state error feedback control law, and an extended state observer. A transition process and the differentiator are implemented in one module. Total disturbance acting on the motor system is estimated by using an input and an output of a controlled object and an applied control input, and an error and the differential of the error are constructed by using the nonlinear state error feedback control law.

The expression of a nonlinear function ƒal(x, σ, γ) is given as follows:

$$fal ( x , σ , γ ) = \{ \begin{matrix}{\vert x \vert}^{σ} sign ( x ) , \vert x \vert > γ \\ \frac{x}{{γ}^{{}_{1 - σ}}} , \vert x \vert \leq γ\end{matrix} ; \qquad ( 1 )$$

wherein, x is an input with error information; σ controls the function to avoid saturation when the value of x is large, 0<σ<1; and γ guarantees that the function may not assume large values in the vicinity of zero for small values of x.

In the present embodiment, steps of the active disturbance rejection controller are as follows:
step 201: based on the inputted rotational speed deviation ω*-ω, obtain an output value y* of the tracking differentiator by means of the tracking differentiator.

The tracking differentiator is established as follows:

$$\{ \begin{matrix}{e}_{1} = x - {y}^{*} \\ \dot{x} = - k \times fal ( {e}_{1} , {σ}_{0} , {γ}_{0} )\end{matrix} ; \qquad ( 2 )$$

wherein, x is the input of the tracking differentiator, that is, the inputted rotational speed deviation ω*-ω; y* is the output of the tracking differentiator; e₁ is an error signal of the tracking differentiator; k is a speed tracking coefficient; σ₀ and γ₀ are initial values of o and γ; and \dot{x} represents the differential of x.
Step 202: Based on compensation factor b₀ and the observed rotational speed value ω outputted by a speed processing and calculation module, obtain an estimated output z₁ and total disturbance z₂ by means of the extended state observer.

Since a motor driving system that requires to be controlled is a first-order system, a second-order extended state observer requires to be constructed as the observer, which is used for estimating overall disturbance and a real-time updated state variable. The disturbance of the bowl-shaped permanent magnet brushless direct-current motor is estimated in real time by the extended state observer, and the extended state observer may be expressed as:

$$\{ \begin{matrix}{e}_{2} = {z}_{1} - ω \\ {\dot{z}}_{1} = {z}_{2} + {b}_{0} {I}^{*} - {μ}_{1} \times fal ( {e}_{2} , {σ}_{1} , {γ}_{1} ) \\ {\dot{z}}_{2} = - {μ}_{2} \times fal ( {e}_{2} , {σ}_{1} , {γ}_{1} )\end{matrix} ; \qquad ( 3 )$$

wherein, z₁ and z₂ represent the estimated output and the estimated total disturbance; e₂ represents a state error, and b₀ represents the compensation factor; μ₁ and μ₂ represent a correction gain of an output error factor; I* is the output of the active disturbance rejection controller, and σ₁ and γ₁ are initial values of σ and γ in the extended state observer.
Step 203: Subtract the output z₁ of the extended state observer from the output y* of the tracking differentiator to obtain e, that is, e=y*−z₁; then obtain a nonlinear control law output u₀ by means of the nonlinear state error feedback control law; and then subtract the total disturbance z₂ from the nonlinear control law output u₀ and divided by b₀ to obtain a compensated output I* of the active disturbance rejection controller.

The nonlinear state error feedback control law is responsible for adjusting the compensation of a controlled variable and improving the effectiveness of feedback control. The nonlinear state error feedback control law input is a difference between the tracking differentiator and the extended state observer, and its expression is as follows:

$$\{ \begin{matrix}e = {y}^{*} - {z}_{1} \\ {u}_{0} = {μ}_{3} \times fal ( e , {σ}_{2} , {γ}_{2} ) \\ {I}^{*} = ( {u}_{0} - {z}_{2} ) / {b}_{0}\end{matrix} ; \qquad ( 4 )$$

wherein, u₀ represents the nonlinear control law output, and mainly depends on the sampling time of a control system; μ₃ represents the correction gain of the output error factor; and σ₂ and γ₂ are initial values of σ and γ in the nonlinear state error feedback control law.

Based on the compensated output I* of the active disturbance rejection controller, under the action of external disturbance d brought to the motor by friction caused by blood flow, the observed rotational speed value ω is obtained by means of the motor system.

For the back electromotive force detection in step 6, a non-conductive phase is selected, and at this time, a voltage measured at both ends of the phase is the back electromotive force.

For the speed processing and calculation of step 6, the back electromotive force value e_{abc} is known, because the relationship between the back electromotive force e_{abc} of the bowl-shaped permanent magnet brushless direct-current motor and the motor rotation speed ωₑ is as follows:

$${e}_{a b c} = {K}_{e} {ω}_{e} ; \qquad ( 5 )$$

wherein, Kₑ is a back electromotive force constant. The magnitude of the back electromotive force can be estimated according to the amplitude of a linear back electromotive force of the observer. Therefore, the estimated speed, that is, the observed rotational speed value ω is as follows:

$$ω = \frac{{ω}_{e}}{p} = \frac{{e}_{a b c}}{p {K}_{e}} ; \qquad ( 6 )$$

wherein, p is the number of pole pairs of the motor. The observed rotor position value θ is as follows:

$$θ = \int ω dt + {θ}_{0} ; \qquad ( 7 )$$

wherein, θ₀ is the initial position angle of the rotor.

The control method for the bowl-shaped permanent magnet brushless direct-current motor provided in the present embodiment has the following beneficial effects:
(1) Strong disturbance rejection capacity: active disturbance rejection control implements very strong disturbance rejection capacity by estimating and compensating for the disturbances in the bowl-shaped permanent magnet brushless direct-current motor. For the bowl-shaped permanent magnet brushless direct-current motor, the active disturbance rejection control can more effectively suppress the impact of external disturbance, such as a voltage fluctuation and a load change, on the bowl-shaped permanent magnet brushless direct-current motor, thereby improving stability and robustness of the bowl-shaped permanent magnet brushless direct-current motor.
(2) Faster response speed: the active disturbance rejection control can implement a faster response speed by employing a more advanced control policy and technology. In the control of the bowl-shaped permanent magnet brushless direct-current motor, a desired speed, position, or torque control can be implemented more quickly, thereby improving the dynamic performance of the bowl-shaped permanent magnet brushless direct-current motor.
(3) No accurate model required: the active disturbance rejection control has a relatively low requirement on an accurate model of the bowl-shaped permanent magnet brushless direct-current motor. In such a complex system of the bowl-shaped permanent magnet brushless direct-current motor, it is often difficult to accurately establish a system model. Therefore, the active disturbance rejection control is more flexible in actual application.
(4) Easy implementation and adjustment: a control structure of the active disturbance rejection control is relatively simple, and parameter adjustment is relatively intuitive. In contrast, proportional-integral control may require more debugging and optimization to achieve the ideal control effect.

### Example 3

The present embodiment provides an artificial heart pump employing the bowl-shaped permanent magnet brushless direct-current motor according to Example 1.

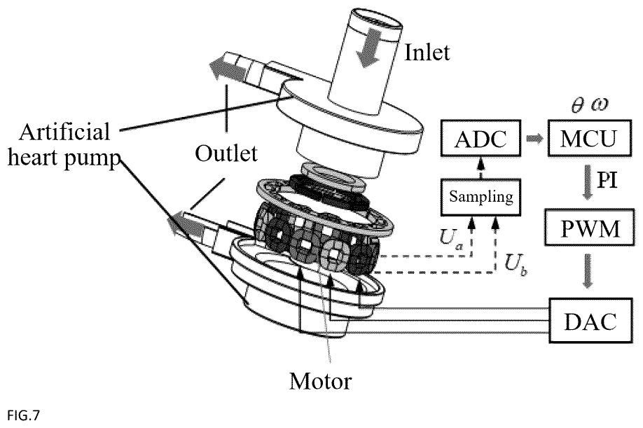

FIG. 7 is a schematic diagram of an artificial heart pump. After a voltage of the bowl-shaped permanent magnet brushless direct-current motor according to Example 1 is sampled, a signal is inputted into a microcontroller unit (MCU) by means of an analog-to-digital converter (ADC) to obtain a back electromotive force zero-crossing signal, and then an observed rotor position value θ and an observed rotational speed value ω are obtained by calculation. Further, an active disturbance rejection controller and a hysteresis current controller calculate each control voltage reference value. A reference voltage signal is inputted into a three-phase inverter bridge, and then is fed to each winding by means of a digital-to-analog converter (DAC) to drive the artificial heart pump to rotate, so that the blood flows in from an inlet and flows out from an outlet.

The foregoing descriptions are only preferred embodiments of the present disclosure and are not intended to limit the present disclosure. For those skilled in the art, the present disclosure may have various changes and variations. Any modification, equivalent replacements, improvements, and the like made within the spirit and principles of the present disclosure shall be included within the scope of protection of the present disclosure.

1. A bowl-shaped permanent magnet brushless direct-current motor, comprising a bowl-shaped stator core, a rotor core, and a plurality of permanent magnets;
wherein the bowl-shaped stator core comprises a stator yoke portion and a plurality of bent teeth, a height difference exists between a tooth shoe of the bent tooth and a plane on which the stator yoke portion is located, and the tooth shoe of the bent tooth is located at the same height as the permanent magnet in an axial direction, so that an armature magnetic flux path is located at the same height as a permanent magnet excitation magnetic flux path;
the permanent magnet is a non-uniform thickness permanent magnet; a radial outer edge of the non-uniform thickness permanent magnet is an arc, a circle center of the outer edge arc is on a rotation axis of the motor; a radial inner edge of the non-uniform thickness permanent magnet is also an arc, a circle center of the inner edge arc is not on the rotation axis of the motor, and a radius of the inner edge arc is less than a maximum distance between any point of the inner edge and the circle center of the outer edge arc;
a radial outer edge of the rotor core coincides with the radial inner edge of the non-uniform thickness permanent magnet to form a plum blossom-shaped structure; and
the non-uniform thickness permanent magnets are evenly distributed in a circumferential direction of the rotor core, and adjacent non-uniform thickness permanent magnets are alternately magnetized inwards and outwards in a radial direction.

2. The bowl-shaped permanent magnet brushless direct-current motor according to claim 1, further comprising a stator winding, wherein the stator winding is wound around the bent teeth.

3. A method for controlling the bowl-shaped permanent magnet brushless direct-current motor according to claim 1, comprising the following steps:
calculating a control voltage reference value by means of an active disturbance rejection controller and a hysteresis current controller, and controlling the bowl-shaped permanent magnet brushless direct-current motor by a three-phase inverter.

4. The method for controlling the bowl-shaped permanent magnet brushless direct-current motor according to claim 3, wherein the active disturbance rejection controller obtains an output of the active disturbance rejection controller based on a deviation between a given rotational speed value and an observed rotational speed value.

5. The method for controlling the bowl-shaped permanent magnet brushless direct-current motor according to claim 4, wherein steps of calculating the observed rotational speed value comprise:
acquiring a voltage output value and a current output value;
based on the voltage output value and the current output value, obtaining a back electromotive force value by means of back electromotive force detection; and
based on the back electromotive force value, obtaining the observed rotational speed value by means of speed processing and calculation.

6. The method for controlling the bowl-shaped permanent magnet brushless direct-current motor according to claim 3, wherein the active disturbance rejection controller estimates disturbance of the bowl-shaped permanent magnet brushless direct-current motor by employing an extended state observer.

7. An artificial heart pump, comprising the bowl-shaped permanent magnet brushless direct-current motor according to claim 1.
